(19) United States
(12) Patent Application Publication
(10) Pub. No.: US 2025/0146040 A1
(43) Pub. Date: May 8, 2025

(54) METHOD OF ENHANCED SINGLE-CELL PROTEIN PRODUCTION FROM VEGETABLE WASTE USING YEAST CO-CULTURE

(71) Applicant: Qatar Foundation for Education, Science and Community Development, Doha (QA)

(72) Inventors: Kashif Rasool, Doha (QA); Hafiz Muhammad Aamir Shahzad, Doha (QA); Khaled Mahmoud, Doha (QA)

(21) Appl. No.: 18/923,291

(22) Filed: Oct. 22, 2024

Related U.S. Application Data
(60) Provisional application No. 63/547,609, filed on Nov. 7, 2023.

(51) Int. Cl. C12P 21/00

(52) U.S. Cl. CPC C12P 21/00; C12P 2201/00

(57) ABSTRACT

The disclosed technology proposes a method of enhanced single-cell protein production from vegetable waste using yeast co-culture. A method of Single Cell Protein (SCP) production including co-culturing Saccharomyces cerevisiae and Candida tropicalis, pretreating vegetable waste, and supplementing the vegetable waste with a nutrient supplement.

### RELATED APPLICATIONS

The present disclosure claims priority to U.S. Provisional Patent Application 63/547,609 having a filing date of Nov. 7, 2023, the entirety of which is incorporated herein.

### BACKGROUND

The Food and Agriculture Organization (FAO) of the United Nations has reported that almost 1.3 billion tons of food, around one-third of the global food production, are wasted annually resulting in estimated costs of $1 trillion. Fruits and vegetables, which are originated from plants, contribute significantly to agro-industrial losses. Approximately 13% of food and vegetables products are wasted during various steps of the production process, including production, storage, and transportation. Additionally, 17% of resources are wasted at the residential, retail, and food service levels. A recent study conducted by the United Nations Environmental Program (UNEP) in Doha revealed that food waste within the city varied from 0.7 kg to 1.5 kg/person/day, adding to the solid waste transported to landfills in the country. These losses have significant consequences on food security, economy, and natural resources. However, these food and vegetable wastes, rich in biologically active compounds, have been increasingly explored as a valuable source for the production of methane, organic acids, ethanol, biodiesel, enzyme, secondary metabolites, and single cell protein (SCP).

In the past few decades, there has been growing interest in the Single Cell Protein (SCP) production, with a focus on utilizing diverse food and vegetable wastes as substrates. The cost and economic feasibility of SCP production are heavily influenced by the cost of the substrate. Therefore, waste derived from different agro-industrial sources presents promising potential as a viable substrate for production of SCP.

Single cell protein refers to the dried cells of microorganisms that are used as a protein source for both the human food and animal feed applications. These cells are obtained by the extraction of proteins from pure cultures or co-cultures of bacteria, fungi, yeasts and microscopic algae. While SCPs are primarily used as supplements in animal feed, their application as a food source for human remains relatively limited. SCPs comprise various components including carbohydrates, fats, vitamins, nucleic acids, and minerals. Notably, SCPs are abundant in essential amino acids such as lysine and methionine, which are often deficient in conventional plant and animal-based diets. While various microorganisms including bacteria, fungi, yeast and algae are commonly used for the production of SCPs, yeast-based SCPs distinguish themselves as a valuable alternative due to their nutritional richness. Yeast possesses several advantages as the preferred microorganism for SCP production, such as its larger size compared to bacteria, significant level of malic acid and lysine, its ability to thrive in acidic pH and its extensive history of traditional use. Research has shown that the most effective approach to efficiently convert carbohydrate wastes into substantial yields of microbial protein within shorter fermentation durations involves the co-cultivation of microorganisms.

The production of SCPs involves certain essential requirements, such as by supplementing nutrient source and by pretreatment with diluted acid (hydrochloric acid, sulfuric acid), alkalinity, thermal hydrolysis and enzymes to assess their effectiveness in generating biomass with a high protein content. The choice of nutrient source can influence environmental conditions, which may, in turn, impact yeast growth and the aim of pretreatment is to convert the complex structure of available nutrients in waste materials into a simpler form that can be easily utilized by microorganisms, thus accelerating the subsequent fermentation process. Among the various pretreatment techniques, hydrolyzing with diluted acid is the most popular pre-treatment method. The acid hydrolysis method is utilized to break down lignocellulose at high temperatures. This process results in the release of lignin, hydrolysis of hemicellulose into individual sugar units, and the formation of a diverse mixture of compounds that can hinder fermentation. Conversely, cellulose remains relatively unaffected and is not easily undergo hydrolysis.

To attain the best possible yields of fermented products, it is important to optimize both pretreatment and cultural conditions, either in pure or co-culture forms. This optimization is aimed to enhance the efficiency of the fermentation process, thereby reducing cost and expediting production. Response surface methodology (RSM) proves to be a valuable tool for optimizing the pretreatment process as it allows for the rapid generation of accurate conclusions. RSM employs mathematical equations that take multiple variables affecting the desired response. One of the significant advantage of RSM is its ability to achieve optimal results with minimal number of experimental runs. The results obtained from this optimization process serve as the foundation for designing large-scale fermenters.

### SUMMARY

According to one non-limiting aspect of the present disclosure, an exemplary embodiment of a method of Single Cell Protein (SCP) production including co-culturing Saccharomyces cerevisiae and Candida tropicalis, pretreating vegetable waste with 4% sulfuric acid at 140° C., and adding nutrient supplement to the vegetable waste.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows an overview of the disclosed technology, according to an example embodiment of the present disclosure.

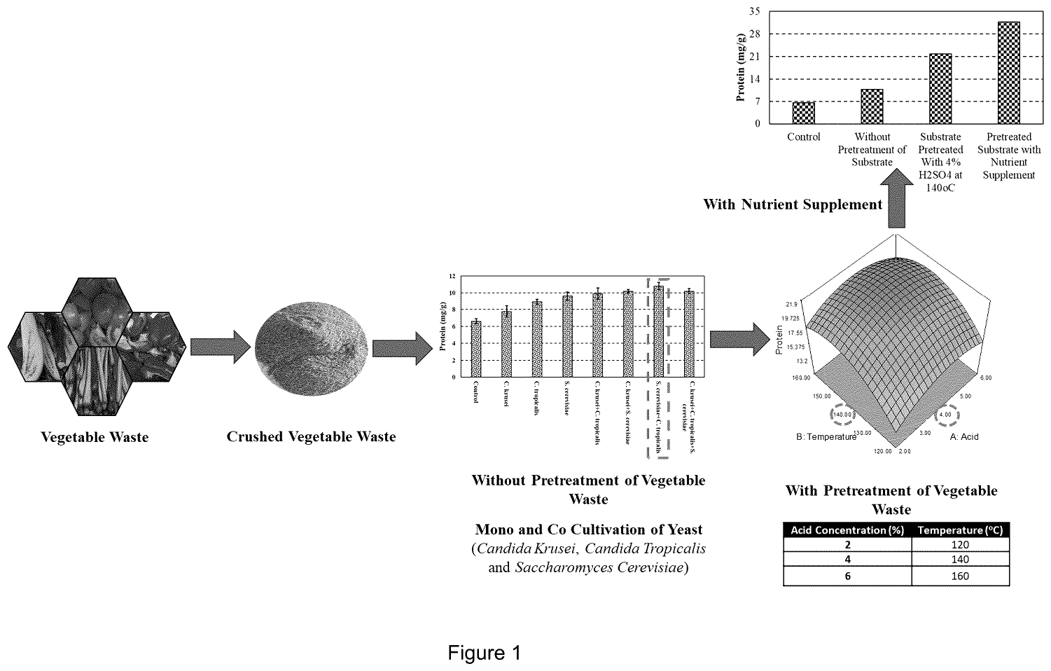

FIG. 2 shows Single Cell Protein (SCP) production from without pretreated vegetable waste using the fermentation of mono and co cultivation of yeast strains, according to an example embodiment of the present disclosure.

FIG. 3 shows the estimated response surface for the SCP production using pretreated vegetable waste, according to an example embodiment of the present disclosure.

FIG. 4 shows the estimated response surface for the reducing sugar content using pretreated vegetable waste, according to an example embodiment of the present disclosure.

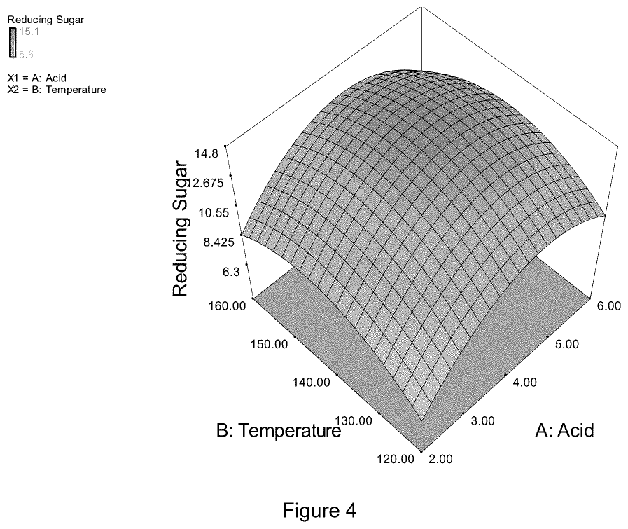

FIGS. 5A-5B show plots of predicted versus actual values for the (a) Single Cell Protein (SCP) production and (b) reducing sugar content, according to an example embodiment of the present disclosure.

FIG. 6 shows Single Cell Protein (SCP) production by using yeast combination of Saccharomyces cerevisiae with Candida tropicalis, according to an example embodiment of the present disclosure.

### DETAILED DESCRIPTION

The present disclosure generally relates to a method of enhanced single-cell protein production from vegetable waste using yeast co-culture.

The disclosed technology proposes to produce single-cell protein (“SCP”) via yeast fermentation using mixture of agro-industrial wastes that have not been explored before, particularly vegetable waste such as tomato, capsicum, eggplant and cucumber. The disclosed technology selected widely used yeasts strains, such as Candida tropicalis, Candida krusei and Saccharomyces cerevisiae, for SCP production from locally produced vegetable waste. Following, the identification of best performing yeast, acid and thermal hydrolysis pretreatment of vegetable waste was optimized using Response surface methodology (“RSM”) progressively to enhance reducing sugar content and SCP yield. Response Surface Methodology (RSM) is a valuable tool for optimizing variable combinations, allowing for the rapid generation of accurate conclusions. It is employed during the acid and thermal hydrolysis pretreatment process to efficiently identify the optimal conditions that enhance SCP production. Subsequently, after the pretreatment optimization of vegetable waste, it was supplemented with nutrient source. Supplementing nutrient in the vegetable waste can stimulate the growth of SCP or enhance the efficiency of the fermentation process. Outlined below is a sample method of enhanced single-cell protein production from vegetable waste using yeast co-culture.

Specifically, the production of single-cell protein (SCP) from vegetable waste represents a promising approach for efficient waste management. In the disclosed technology, SCP was generated through solid state fermentation using vegetable waste, namely tomato, capsicum, eggplant and cucumber. In an associated study, three yeast strains (Candida tropicalis, Candida krusei and Saccharomyces cerevisiae) were selected to obtain a high-value of SCP by optimizing cultural conditions without pretreatment of substrate. Following, the identification of best performing yeast acid and thermal hydrolysis pretreatment of vegetable waste was optimized using central composite design by RSM. Pretreatment of the substrate was achieved with varying concentrations of sulfuric acid (2, 4 and 6%) at the temperature of 120, 140 and 160° C. Subsequently, after the pretreatment optimization, vegetable waste was supplemented with nutrient source. Without pretreatment of vegetable waste, a maximum protein content of 10.8 mg/g of dry biomass (which is an increment of 63.6% as compared to control) was achieved with the yeast combination of Saccharomyces cerevisiae and Candida tropicalis. With the same yeast strains, pretreatment of vegetable waste (with 4% sulfuric acid at 140° C.) and supplemented with nutrient source increased SCP production to 21.9 and 31.7 mg/g (which is the increment of 231.8 and 375.8% as compared to control), respectively. Thus, the study demonstrated that SCP production can be enhanced with the acid and thermal hydrolysis of nutrient supplemented vegetable waste by using optimized cultural conditions.

### Sample Collection and Preparation of Fermentation Medium

Vegetable waste past its expiration date such as tomato, capsicum, eggplant and cucumber were collected from supermarkets and grocery stores Doha, Qatar. The proximate analysis of the collected vegetable waste involved determining the moisture, ash, lipid, carbohydrate, and protein contents is summarized in Table 1. Ash (A), protein (P), and lipid (L) content were analyzed using methods specified by AOAC. The determination of total carbohydrates (TC) was achieved through a weight difference calculation using the following equation: TC=100−(A+P+L)

Prior to its use, the collected vegetable waste was thoroughly washed and dried at 60° C. for 48 hours in a conventional oven (Heratherm OMS60, Thermo Scientific, Germany). Substrate was then crushed using impact grinding (HR7778, Philips, China) and sieved to achieve a particle size of less than 1 mm.

TABLE 1
Proximate analysis of the vegetable waste.

| Constituent | Unit | Value |
|---|---|---|
| Moisture Content (Fresh) | % | 93.1 ± 0.02 |
| Ash Content | % | 11.06 ± 0.08 |
| Protein Content | % | 0.66 ± 0.03 |
| Lipid Content | % | 3.24 ± 0.04 |
| Carbohydrates | % | 85.04 ± 0.15 |

### Inoculum Preparation

Three strains (Candida tropicalis, Candida krusei and Saccharomyces cerevisiae) were obtained. The cultures were maintained on yeast extract peptone dextrose (YPD) medium. All yeasts were cultured at 28° C. for 72 hours and stored at 4° C. The strain was stored in 30% (w/v) glycerol at a temperature of −80° C. The yeast was reactivated by transferring of 1 mL of cell suspension into a 50 mL flask containing 30 mL YPD medium at 28° C. for 48 hours and cultures were used as inoculum for SCP production. The cell suspension was adjusted to a final concentration of 1×10⁶ CFU/mL.

### Fermentation Process

The disclosed technology is divided into three different phases, as outlined in Table 2. In phase 1, 10 g of substrate was transferred in each petri dish and the cultural conditions of yeast strains, in pure and co-culture forms, were optimized without pretreatment of vegetable waste substrate for the production of SCP. All assays were carried out in duplicates. Means and standard deviations (SD) were determined using SPSS version 23. Controls without inoculum were also incubated under the same fermentation conditions. Following the identification of best performing yeast cultural conditions, phase 2 involved optimization of acid and thermal hydrolysis pretreatment of vegetable waste by using RSM. For pretreatment, 10 g of substrate in each petri dish was hydrolyzed with 2, 4 and 6% of H₂SO₄ maintaining a liquid to solid ratio of 6:4. Hydrolysis was carried out at 120, 140 and 160° C. for 1 hour and then was allowed to cool. After thermal pretreatment, a moisture content of 60% was adjusted by addition of distilled water before autoclaving for 15 minutes at 121° C. and pH of the substrate was adjusted to 5.0 by using 1 N NaOH solution. Subsequently, after the pretreatment optimization, pretreated vegetable waste was supplemented with nutrient source containing KH₂PO₄ (1 g), MgSO₄·7H₂O (0.5 g), NaCl (0.1 g) and CaCl₂ (0.1 g) made up to 1 L and used with pretreatment hydrolysis maintaining a liquid to solid ratio of 6:4.

TABLE 2
Various phases of the study.

| Phase 1 | | | | | Phase 3 | |
|---|---|---|---|---|---|---|
| Optimization of | | Phase 2 | | | Supplementation of | |
| cultural conditions | | Optimization of pretreatment of substrate | | | nutrient source | |
| Cultural | | Cultural | Pretreatment of substrate | | Cultural | |
| conditions | | conditions | Acid | | conditions | |
| of yeast | Pretreatment | of yeast | Concentration | Temperature | of yeast | Pretreatment |
| strains | of substrate | strains | (%) | (° C.) | strains | of substrate |
| Mono and | Without | Optimized | 2 | 120 | Optimized | Optimized |
| mixed | pretreatment | cultural | 4 | 140 | cultural | pretreatment |
| cultivation | of substrate | conditions | 6 | 160 | conditions | conditions |
| of yeast | | from phase 1 | | | from | from phase |
| | | | | | phase 1 | 2 |

After sterilization, substrate in each petri dish was inoculated with 2 mL of yeast strain inoculum. The fermentation was carried out in the shaking incubator (MaxQ 800, Thermo Scientific, Netherlands) at a speed of 100 rpm for 5 days at 28±2° C. followed by determination of protein content.

### Response Surface Methodology Experimental Design

Response surface methodology (RSM) was utilized for the optimization of pretreatment conditions (acid concentration and temperature) by evaluation with a three-level factorial model design for reducing sugar content and SCP Yield. Two dependent variables, the concentration of sulfuric acid (2, 4 and 6%) and temperature (120, 140 and 160° C.) were selected in this study for optimization while the observed response was the reducing sugar and protein content of fermented vegetable waste.

The treatment combinations were analyzed according to the central composite design using Design-Expert statistical software (version 7.0.0, STATEASE Inc, Minneapolis, MN, USA). The validity of the model was expressed as a regression coefficient (R²), and the significance of the regression coefficients was evaluated by analysis of variance (ANOVA).

The design of the experiment associated with the disclosed technology, including the dependent variables, is shown in Table 2.

TABLE 3
Experimental design of the optimization study (phase 2).
Run

| | | | |
|---|---|---|---|
| Factor | −1 | 0 | 1 |
| Acid Concentration | 2% | 4% | 6% |
| Temperature | 120° C. | 140° C. | 160° C. |

### Analytical Methods

After 5 days of fermentation, samples were dried, grinded and homogenized for measurement of protein content. Protein enrichment in dry biomass due to increase in microbial population from each assays was determined on the basis of nitrogen content (%) using kjeldahl method by multiplying conversion factor of 6.25. Approximately 1 g of the substrate was subjected to hydrolysis with 15 mL of concentrated sulfuric acid (H₂SO₄), which contained two copper catalyst tablets. This hydrolysis process took place in a heat block (KjelDigester k-449, Buchi, Switzerland) and was digested at 420° C. for a duration of 2 hours. After cooling, distillation and titration were performed using KjelMaster K-375 (Buchi, Switzerland). In the process, the liberated ammonium was collected through distillation and captured in a boric acid solution. Following this, titration with H₂SO₄ was performed to determine the nitrogen present in the samples.

Phase 1: Optimization of Cultural Conditions and Production of SCP without Pretreatment of Vegetable Waste

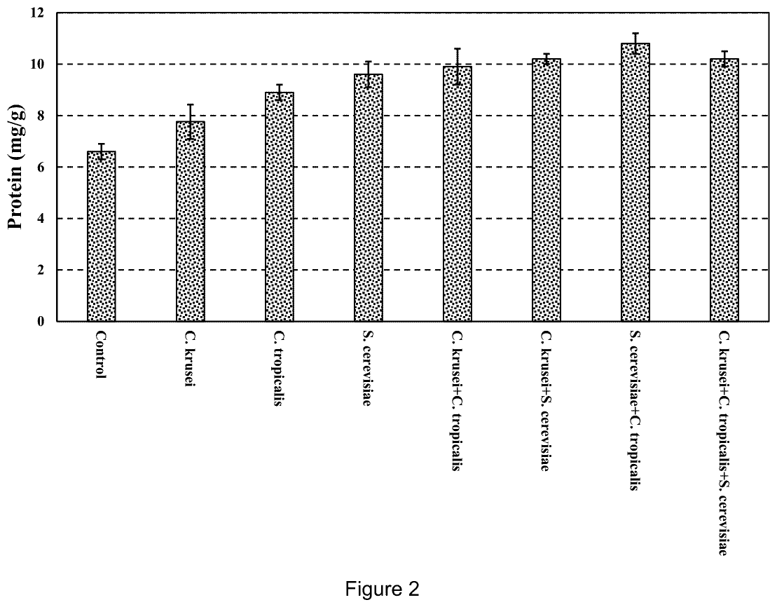

In the first phase of the study, the fermentation test on without pretreated vegetable waste with mono and co-culture of yeasts (Candida tropicalis, Candida krusei and Saccharomyces cerevisiae) was carried out for SCP production and illustrated in FIG. 2. The results indicated that, under control conditions where no inoculum was applied, the SCP production yielded 6.6 mg/g of dry biomass. However, when the substrate was fermented with mono yeast strains of Candida tropicalis, Candida krusei and Saccharomyces cerevisiae the SCP production increased to 8.9, 7.6 and 9.6 mg/g of dry biomass, respectively. Which is the increment of 34, 17 and 46% in SCP production with yeast strains Candida tropicalis, Candida krusei and Saccharomyces cerevisiae over the control, respectively. It is widely recognized that each yeast species exhibits varying abilities and preferences when it comes to utilizing different types of substrates. For instance, Saccharomyces cerevisiae proficiently metabolizes glucose, sucrose, and maltose, while it remains incapable of utilizing lactose and soluble starch. Similarly, Candida tropicalis efficiently consumes sucrose, maltose, and glucose as carbon sources but shows limited or no capacity for lactose and soluble starch utilization, aligning with Saccharomyces cerevisiae. On the other hand, Candida krusei displays a robust proficiency in breaking down starch, dxylose, and hydrocarbon organic matter. Notably, Candida krusei thrives in acidic and high-temperature environments, yielding superior outcomes at low pH levels (3.5) and elevated temperatures (40-43° C.).

However, it is worth noting that the Saccharomyces cerevisiae exhibited the highest SCP yield of 9.6 mg/g of dry biomass compared to other yeast strains (FIG. 2). The higher crude protein, approximately up to 62% observed in Saccharomyces cerevisiae. On the other hand, Candida tropicalis and Candida krusei has lower protein content around 60% and 47%, respectively resulting in a lower SCP production.

In mixed or co cultivation of yeast strains test these yeast strains combined to each other, in combination of two and three, to analyze their ability for SCP production. As depicted in FIG. 2, SCP production of 9.9, 10.2 and 10.8 mg/g of dry biomass was achieved when substrate was fermented with combination of Candida tropicalis and Candida krusei, Saccharomyces cerevisiae and Candida krusei and Saccharomyces cerevisiae and Candida tropicalis, respectively. Subsequently, Saccharomyces cerevisiae, Candida krusei and Candida tropicalis were employed in three yeast fermentation trials and SCP production of 10 mg/g of dry biomass was achieved. The highest SCP production was achieved when combining Saccharomyces cerevisiae with Candida tropicalis which is the increment of 63.6% as compared to control. Therefore, this yeast combination considered as the optimal strain for production of SCP from vegetable waste.

As depicted in FIG. 2, the batch culture of mono and mixed culture results, mixed culture resulted in more production of protein content. This aligns with previous research, where mixed cultures consistently outperformed single-culture methods in SCP production. This phenomenon can be attributed to the symbiotic relationships that develop between different yeast strains when working in combination.

### Phase 2: Optimization of Pretreatment Parameters of Vegetable Waste for Production of SCP

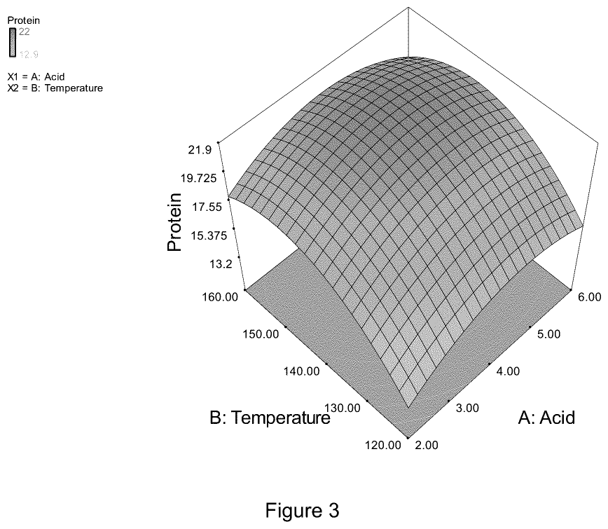

Yeast strains used in this study lack the enzyme system required for the hydrolysis of polysaccharides into monosaccharides and resulted in low protein production in previous phase. The vegetable waste was transformed into fermentable sugars for the production of SCP through acid and heat treatment process. The central composite design proved effective in assessing protein production using pretreated vegetable waste, with acid concentration and temperature as key factors. The fermentation experiments with the combination of Saccharomyces cerevisiae and Candida tropicalis (optimized in previous phase) were conducted at 28° C. for a duration of 5 days. FIGS. 2 and 3 illustrate the SCP production and reducing sugar content by mixed cultures of yeast (Saccharomyces cerevisiae with Candida tropicalis) strains while utilizing pretreated vegetable waste. In general, it was observed that the reducing sugar content and SCP production was increased as the acid concentration during pretreatment was increased from 2% to 4%. However, with further increase in acid concentration the SCP production and reducing sugar content are decreased as depicted in FIGS. 2 and 3. This observation could be attributed to the occurrence of additional oxidation of reducing sugars as the acid concentration further increased. Temperature also plays a critical role in biochemical reactions, as the rate constant of hydrolysis reactions is temperature dependent. Powdered vegetable waste was subjected to hydrolysis using H₂SO₄ at different temperatures, specifically 120° C., 140° C., and 160° C. When the temperature increases from 120 to 140° C. the reducing sugar content and SCP production with each yeast strain was increased. However, as the temperature increased to 160° C. the SCP production slightly decreased as shown in FIG. 2. As reported earlier, within the temperature range of 60 to 140° C., all nutrients content in vegetable waste increased with temperature resulted in increase in SCP production.

Based on optimization using RSM, the SCP production of fermented vegetable waste of 21.9 mg/g of dry biomass with highest sugar content of 14.6% was obtained with pretreatment of substrate at temperature of 140° C. with 4% H₂SO₄ concentration. Which is the increment of 102.7% in SCP production as compared to without pretreated vegetable waste by using similar yeast strains. The significance of the model was assessed using ANOVA, and the validity of the model equations was determined through the F-test, as presented in Table 4. For the SCP production with pretreatment of vegetable waste, the model exhibited an F-value of 41.68. The model's probability value for this process was less than 0.0001, indicating that the model terms for all the experimental conditions held significant importance. The R² value obtained from the model (refer to Table 4) for SCP production was 0.97, which is very close to one. This indicates that the model has the potential to describe up to 97% of the variation. Additionally, it demonstrates a strong alignment between the model's predictions and the experimental values, as evidenced by the low coefficient of variation (CV) of 4.27%. Moreover, the lack-of-fit value was found to be insignificant (>0.05), further corroborating the model's goodness of fit. The results clearly show that this model accurately describes and predicts SCP production.

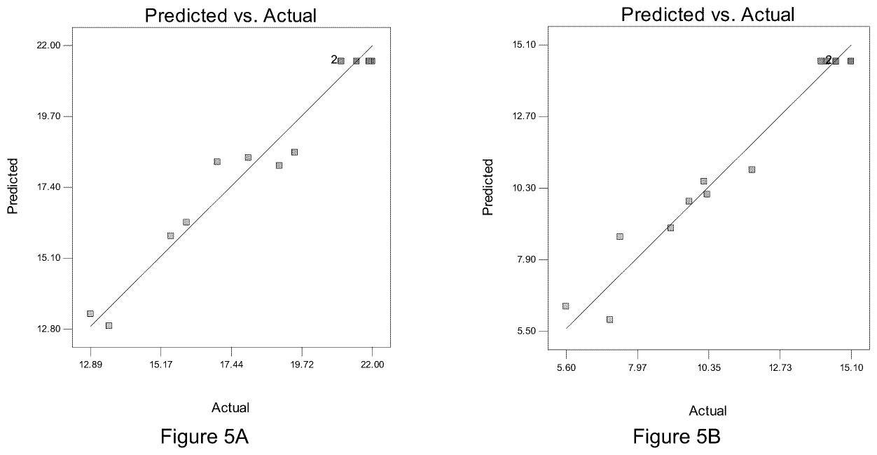

In FIG. 5, the predicted points versus the actual plots for the reducing sugar and SCP production align closely along the diagonal line, indicating that the predicted values closely match the observed values. To explore the significance of the acid and thermal pretreatment effects on SCP production, the ANOVA data presented in Table 4 were analyzed. In the experiment conducted, the statistical analysis identified acid and temperature as the most critical pretreatment factors in achieving a high SCP production (where Prob>F values of less than 0.05 indicated the significance of the model term).

TABLE 4
ANOVA model of SCP production for pretreatment optimization.

| Source | Df | Mean Square | F-value | p-value Prob > F |
|---|---|---|---|---|
| Model | 5 | 24.47 | 41.68 | <0.0001 |
| A-Acid Concentration | 1 | 1.54 | 2.63 | 0.1490 |
| B-Temperature | 1 | 11.63 | 19.80 | 0.0030 |
| AB | 1 | 2.40 | 4.09 | 0.0828 |
| A₂ | 1 | 38.70 | 65.91 | <0.0001 |
| B₂ | 1 | 80.83 | 137.64 | <0.0001 |
| Residual | 7 | 0.59 | | |
| Lack of Fit | 3 | 1.07 | 4.70 | 0.0844 |
| Pure Error | 4 | 0.23 | | |
| Core Total | 12 | | | |
| R² | 0.97 | | | |
| C.V. % | 4.27 | | | |

It can be evident that the acid and thermal pretreatment of vegetable waste is the most influential variables and had a positive influence on SCP production. Similar findings showed acid and thermal pretreatment of vegetable waste at different concentrations stimulated SCP production. The growth of yeast strains increased with increased in the reducing sugar concentration with acid and thermal pretreatment of vegetable waste resulting in increased in protein production by yeast. These findings align with another study, which also demonstrated that the acid and thermal hydrolysis of vegetable waste enhanced the accessibility of carbon sources within vegetable waste, encompassing residues from vegetable waste by deconstructing intricate carbohydrates into low molecular weight dextrin and glucose resulted in increase in SCP production. Thus, pretreatment may have a crucial importance in production of SCP by yeast strains as shown by the low protein content in without pretreated vegetable waste.

### Phase 3: Effect of Nutrient on SCP Protein Optimized Pretreatment and Co-Culture Conditions

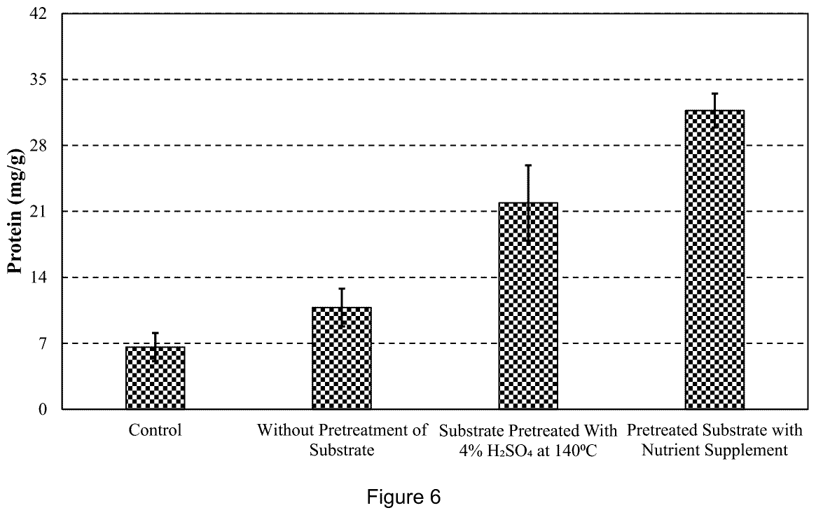

With a low SCP production of 21.9 mg/g of dry biomass from vegetable waste, the impact of nutrient supplementation on protein enrichment was examined. As shown in FIG. 6, nutrient supplementation was found to be the effective in enhancing SCP production using pretreated vegetable waste at temperature of 140° C. with 4% H₂SO₄ concentration fermented by co culture of yeast strain Saccharomyces cerevisiae with Candida tropicalis. This supplementation resulted in an increase of SCP production of 31.7 mg/g of dry biomass, which is the increment of 44.74%. The protein content in the final pretreated and nutrient supplemented fermentation product represents an increment of 375.8% from the original protein content of the unfermented vegetable waste (control). These experimental findings align with previous studies where protein enrichment was observed when substrates were supplemented with nutrients.

The production of single-cell protein (SCP) from vegetable waste represents a promising approach for efficient waste management. In the current study, SCP was generated through solid state fermentation using vegetable waste, namely tomato, capsicum, eggplant and cucumber. Three yeast strains (Candida tropicalis, Candida krusei and Saccharomyces cerevisiae) were selected to obtain a high-value of SCP by optimizing cultural conditions without pretreatment of substrate. Following, the identification of best performing yeast acid and thermal hydrolysis pretreatment of vegetable waste was optimized using central composite design by response surface methodology. Pretreatment of the substrate was achieved with varying concentrations of sulfuric acid (2, 4 and 6%) at the temperature of 120, 140 and 160° C. Subsequently, after the pretreatment optimization, vegetable waste was supplemented with nutrient source. Without pretreatment of vegetable waste, a maximum protein content of 10.8 mg/g of dry biomass (which is an increment of 63.6% as compared to control) was achieved with the yeast combination of Saccharomyces cerevisiae and Candida tropicalis. With the same yeast strains, pretreatment of vegetable waste (with 4% sulfuric acid at 140° C.) and supplemented with nutrient source increased SCP production to 21.9 and 31.7 mg/g (which is the increment of 231.8 and 375.8% as compared to control), respectively. Thus, the study demonstrated that SCP production can be enhanced with the acid and thermal hydrolysis of nutrient supplemented vegetable waste by using optimized cultural conditions.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of Single Cell Protein (SCP) production comprising:
co-culturing Saccharomyces cerevisiae (S. cerevisiae) and Candida tropicalis (C. tropicalis);
pretreating vegetable waste; and
supplementing the vegetable waste with a nutrient supplement.

2. The method of claim 1, wherein the S. cerevisiae and C. tropicalis are co-cultured via solid state fermentation using the vegetable waste as a substrate.

3. The method of claim 1, wherein the pretreating includes pretreatment using acid and thermal hydrolysis.

4. The method of claim 3, wherein the acid used in the hydrolysis is sulfuric acid (H₂SO₄).

5. The method of claim 4, wherein the sulfuric acid is at a concentration between 2 and 6%.

6. The method of claim 1, wherein the pretreating using acid and thermal hydrolysis is done between 120° and 160° C. for 1 hour, and subsequently allowed to cool to room temperature.

7. The method of claim 1, further comprising adjusting the pH level of the vegetable to 5.0 after pretreating.

8. The method of claim 7, wherein the pH level is adjusted using 1N NaOH.

9. The method of claim 1, wherein the nutrient supplement comprises KH₂PO₄, MgSO₄·7H₂O, NaCl, and CaCl₂.

10. The method of claim 1, wherein the nutrient supplement comprises 1 gram of KH₂PO₄, 0.5 grams of MgSO₄·7H₂O, 0.1 grams of NaCl, and 0.1 grams of CaCl₂ in a 1 L nutrient supplement.

11. The method of claim 1, further comprising washing and drying the vegetable waste prior to pretreating.

12. The method of claim 11, wherein the vegetable waste is dried at 60° C. for 48 hours in a conventional oven.

13. The method of claim 11, further comprising grinding the vegetable waste after washing and drying and prior to pretreating.

14. The method of claim 13, wherein the vegetable waste is ground to a particle size of less than 1 mm.
